(19) United States
(12) Patent Application Publication
(10) Pub. No.: US 2005/0234148 A1
(43) Pub. Date: Oct. 20, 2005

(54) Agglomerated fillers for dental materials

(75) Inventors: Klaus Ruppert, Maintal (DE); Andreas Grundler, Wuppertal (DE); Albert Erdrich, Bad Nauheim (DE)

Correspondence Address: Kurt G. Briscoe;Norris, McLaughlin & Marcus P.A., 18th Floor, 875 Third Avenue, New York, NY, 10022, US

(73) Assignee: Heraeus Kulzer GmbH, Hanau (DE)

(21) Appl. No.: 11/100,232

(22) Filed: Apr. 6, 2005

(30) Foreign Application Priority Data
Apr. 7, 2004 (DE) 10 2004 017 562.4

(51) Int. Cl. C04B 14/04; A61K 6/08

(52) U.S. Cl. 523/116; 106/489

(57) ABSTRACT

Agglomerated inorganic glass fillers for dental materials comprising 0.5 to 50 μm large agglomerates of inorganic glass particles having a particle size of 200 to 7,000 nm that are fused at their boundary surfaces with at least one adjacent particle, are particularly suitable for dental materials with good polishability, lasting shine and good abrasion-resistance.

The invention concerns agglomerated inorganic fillers for dental materials.

Teeth and also their fillings are exposed to abrasion processes particularly while brushing teeth. In the case of the known composite tooth filling materials comprising organic and strengthening fillers, the abrasion takes place usually in several steps:

First the matrix enveloping the filler particles is removed by an abrasive medium e.g. toothpaste or stone cells in the chyme. The matrix is usually a polymer material e.g. an integrated polymer with the base of methacrylic acid ester.

Eventually the filler particles are so worked out so far from the surface of the polymers that they lose their footing and break out from the surface.

Crevices in the surface (craters) remain behind.

Both the raised filler particles and also the craters lead to diffuse reflection of light in the roughness of the surface, reduce the portion of totally reflected light and also reduce the superficial shine of the originally perfectly polished surface. This partial abrasion process is therefore undesirable due to aesthetic reasons.

The reason for selective abrasion is the essentially higher hardness and abrasion resistance of the filler particles compared to the integrating polymer matrix surrounding them. For avoiding the partial or selective abrasion process a concept was developed that is based essentially on the fact that the particles are not supposed to stick out of the surface anymore. For this purpose agglomerated filler particles were made available that comprise sub-particles with particle sizes in the micrometer or submicron range whose hardness and agglomeration resistance is more similar to that of the surrounding polymer system. Filler materials have already been developed that utilize this principle and contain agglomerated clusters comprising nanoparticles: a tooth filling material containing such agglomerated fillers is the so-called “Filtek™ Supreme Universal Restorative” of the company 3M™ ESPE™.

It essentially consists of a polymer portion with the components Bis-GMA, Bis-EMA, UDMA and small quantities of TEGDMA as well as fillers and is supplied in different color shades.

The translucent, non-radio opaque parts of the assortment of material contain a combination of non-agglomerated/non-aggregated 75 nm silica-nanofiller as well as loosely bound agglomerated silica nanoclusters comprising agglomerates of silica-nano-primary particles (75 nm particle size). The size range of the agglomerates, also referred to as clusters, is 0.6 to 1.4 micrometer. The filling level amounts to 72.5 wt. %.

The non-translucent, radio opaque parts of the assortment contain a combination of non-agglomerated/non-aggregated 20 nm silica-nanofillers as well as loosely bound agglomerated zirconia/silica nanoclusters that are agglomerates of ZrO₂/SiO₂ primary particles with particle sizes of 5-20 nm. The cluster size is again 0.6 to 1.4 micrometer. The filling level amounts to 78.5 wt. % (Product profile Filtek™ Supreme).

The clusters are obtained by thermal treatment (e.g. WO 200130306A1, page 31 and WO 200130304A1, page 7).

The object of the invention is to provide more agglomerated inorganic filler materials. These agglomerates have such a high mechanical stability that they withstand the mechanical stresses during the manufacturing process of dental composites and they are worked out not entirely during the abrasion process, instead only in parts and in layers from the finished composite by selective abrasion.

This task is solved by agglomerating the primary particles made of glass by thermal treatment. That is they melt down superficially with at least one of the adjoining particles. The result is agglomerated inorganic glass fillers for dental materials comprising 0.5 to 50 μm large agglomerates of 200 to 7,000 nm large inorganic glass particles that are fused at their boundary surfaces with at least one adjoining particle.

The particle size is defined via the so-called d 50-value.

The invention thus concerns fillers as described hereinbelow, process for their manufacture, as well as their use in dental materials.

The dental materials can contain additional inorganic fillers, e.g. with particle sizes of 2 to 30 nm and of 30 to 200 nm. Among them are inorganic oxides such as SiO₂, Al₂O₃, ZrO₂, Y₂O₃, particularly precipitated silica and nanofillers as described in e.g. U.S. Pat. No. 5,936,006. The agglomerated glasses and the additional fillers can be surface-modified, particularly silanized, e.g. by treatment with gamma-methacryloxypropyltrimethoxysilane.

The agglomeration takes place by using controlled thermal treatment. The result is that the particles melt together on the boundary surfaces. The result of controlling the treatment time and treatment temperature is that the tensile strength of the particles is so high that they survive the manufacturing process of the dental materials, but so low that during the abrasion process, the particles are removed not entirely, instead in layers and/or in parts. This provides a microscopically smooth surface with lasting, satisfactory shine.

The agglomerated material is advantageously ground to 0.5 to 50 μm large particles, preferably by grinding processes, and if necessary with a subsequent sieving or classifying process.

The invention thus also concerns a process for manufacturing agglomerated inorganic filler materials with the steps:
A preparation of 200 to 7,000 nm large inorganic glass particles by grinding large particles,
B thermal treatment by partial melting at 200 to 1,300° C. (calcination of the glass particles),
C cooling down fast, if necessary,
D grinding the thermally agglomerated material.

Preferably dental glasses are considered as glass material, particularly those that contain chemical elements from the group Ba, Al, Si, O, F, B, Sr, Zr, such as e.g. Ba—, Sr—, Ca—, Li—Al-silicate glasses or mixtures thereof, particularly Li—Al-borosilicate glasses or mixtures thereof as well as barium aluminum borosilicate glass.

The temperatures during the thermal treatment depend on the material and are generally in the range of 200 to 1,300° C.

The starting material can be a dispersion as described in e.g. U.S. Pat. No. 4,503,169, wherein ground glass particles are used. The thermal treatment of the particles can take place in different ways, e.g. directly in a flame or in a hot stream of gas (in accordance with U.S. Pat. No. 5,559,170, columns 15, 16, EP 757 664, claim 29) or by spray drying a dispersion and subsequent calcination (compare U.S. Pat. No. 6,362,251 B1, examples 1-4).

Glass-granulates are also considered that are manufactured analogous to the method of DE 44 24 044 by compaction of a suspension and subsequent calcination/partial melting.

Another possible process is hot pressing in accordance with DE 198 21 679 A1. Thereby preferably particles of two different glasses are used among which one softens at a lower temperature than the other. The glass “melting” at the lower temperature then creates a solder for the glass “melting” at a higher temperature.

Likewise a very fine fraction of the same material can also be used. Here also lower melting temperatures are obtained due to the higher sintering activity.

It is also possible to modify a process in accordance with DE 101 63 179 such that instead of the pyrogenic silica, finely ground glasses are used, or in accordance with DE 196 29 690 C2 and DE 196 0 2525 A1 and/or U.S. Pat. No. 5,858,325 suspensions/slips of glass particles and a solvent are granulated by (fluidized bed)-spray granulation and then subjected to a shock sintering.

After the thermal treatment, the agglomerated particles are advantageously cooled down fast in order to prevent an agglomeration that is too strong.

Example of Manufacture

Barium aluminum silicate glass is finely ground in a mill and sieved. The fraction of approximately 200 to 500 nm particle size is processed further.

A suspension is made by mixing it with water in a blender that results in a free flowing granulate after sedimentation. The granulate is thermally treated at 650 to 950° C., cooled down fast and ground subsequently.

Agglomerate particles with a diameter of 2 to 15 μm are obtained.

1. Agglomerated inorganic fillers comprising agglomerates of inorganic particles fused at their boundary surfaces to at least one adjacent particle, the agglomerates ranging in size from 0.5 to 50 μm, and the inorganic particles having a particle size of 200 to 7,000 nm.

2. Process for preparing fillers in accordance with claim 1, said process comprising the following steps:
A) preparing inorganic particles ranging in size from 200 to 7,000 nm by grinding larger particles,
B) partially melting the inorganic particles by thermal treatment at 200 to 1,300° C. to form a thermally agglomerated material,
C) optionally quickly cooling the thermally agglomerated material, and
D grinding the thermally agglomerated material.

3. Process in accordance with claim 2, wherein the preparation in step A comprises grinding glass.

4. Process in accordance with claim 3, wherein the glass contains chemical elements from the group Ba, Al, Si, O, F, B, Sr, Zr.

5. A method of preparing a dental material, comprising incorporating fillers in accordance with claim 1 into a dental material.

6. Dental material comprising one or more fillers in accordance with claim 1.

7. Dental material in accordance with claim 6, additionally comprising one or more inorganic fillers with particle sizes of 2 to 30 nm and/or 30 to 200 nm.

8. Dental material in accordance with claim 6, wherein the fillers are surface-modified.

9. Dental material in accordance with claim 8 wherein the fillers are silanized.
